(19) United States
(12) Patent Application Publication
(10) Pub. No.: US 2019/0104774 A1
(43) Pub. Date: Apr. 11, 2019

(54) SYSTEM AND METHOD FOR CONTROLLING THE TEMPERATURE OF A SKIN AND/OR MUSCLE SURFACE

(71) Applicants: Bernardo Lembo Conde De Paiva, São Paulo (BR); Raphael Einsfeld Simões Ferreira, São Paulo (BR)

(72) Inventors: Bernardo Lembo Conde De Paiva, São Paulo (BR); Raphael Einsfeld Simões Ferreira, São Paulo (BR)

(21) Appl. No.: 16/087,751

(22) PCT Filed: Mar. 24, 2017
(86) PCT No.: PCT/BR2017/050071

§ 371 Date: Sep. 24, 2018

(30) Foreign Application Priority Data
Mar. 24, 2016 (BR) BR 102016006622-0

(51) Int. Cl. A41D 13/005; A61F 7/02; A61F 7/08; F28F 3/12; F25B 21/02

(52) U.S. Cl. CPC A41D 13/005; A61F 7/02; A61F 7/08; A61F 2007/0236; F25B 21/02; A61F 2007/0234; F28F 3/12

(57) ABSTRACT

A system and method of temperature control for a cutaneous and/or muscle surface by direct and/or indirect contact, having temperature control to aid in the recovery of muscle injuries, improvement in recovery after physical exercise, improvement in performance during physical activities, analgesia, provide greater thermal comfort, assist in aesthetic and postoperative procedures, prevention of alopecia among other solutions related to heating/cooling of the cutaneous and/or muscular surface in humans and other animals by means of heat exchange with a fluid, in the fields of medicine and engineering.

### FIELD OF THE INVENTION

The present invention discloses systems of temperature control for a cutaneous and/or muscle surface by direct and/or indirect contact. The present invention is in the fields of Medicine and Engineering.

### BACKGROUND OF THE INVENTION

It is estimated that at least 20% of the world population practices some type of sport, representing 1.5 billion consumers. Of these, 50 million are categorized as professional and semiprofessional athletes considering the most practiced sports in the world.

The search for performance improvement has always been part of the sports universe and in this context it is evident the need for devices that improve the performance of sportsmen. There is scientific evidence mentioning that muscle cooling can improve athlete performance by up to 20%.

In addition, therapy/treatment of lesions and analgesia of patients by the application of hot or cold wads on the affected site is very effective for the recovery of the lesions and analgesia processes. However, the commonly used methods are uncomfortable and difficult to use, and more modern methods present a high implementation cost.

Cryotherapy, or cold therapy, is a therapy that does the local or general use of low temperatures, being used to treat a variety of injuries in the benign and malignant tissues (lesions). This therapy aims to reduce local inflammation, decrease pain and spasm, promote constriction of blood vessels (vasoconstriction), decrease cell activity and metabolism, thereby decreasing cell damage and increasing cell survival.

Cold therapy is commonly used as a procedure to relieve pain symptoms, especially in inflammatory diseases and overuse injuries and symptoms. A particular form of cold therapy (or stimulation) has been proposed for the treatment of rheumatic diseases. The therapy, called whole-body cryotherapy (WBC), is composed of exposing a patient to very cold air kept at −110° C. and −140° C. in special temperature-controlled chambers, usually for 2 minutes.

WBC is used to relieve pain and inflammatory symptoms caused by numerous disorders, particularly those associated with rheumatic diseases, and is also recommended for the treatment of arthritis, fibromyalgia and ankylosing spondylosis. In sports medicine, WBC has gained wider acceptance as a method to improve recovery from a muscle injury.

Through this therapy, it was concluded that the ability of resistance exercises decreases in warm ambient conditions, however the time until exhaustion can be increased by decreasing body temperature before exercise (precooling).

However, the muscles most used by athletes during their workouts can only be cooled simultaneously through the WBC system, since no other means are available that allow for efficient body cooling by means of a portable system encompassing the muscles used in a single piece.

There are also systems that cool whole body through cooling chambers mainly using liquid nitrogen. This method is extremely exposed to the patient (athlete) and there are several contraindications to its use. The time of a course does not take more than 3 minutes and the internal temperature of the chamber reaches −135° C., which generates some risks such as ischemia and necrosis of distal parts of the limbs, hemodynamic changes such as bradycardia and hypotension, headaches, uncontrollable tremors and even death. In addition, treatment in cooling chambers is a low-accuracy method, as there is no possibility of controlling the target temperature, as well as cooling is in full rather than specific musculature, leading to an greater risk of complications. They are expensive equipment. The investment for the acquisition of a cooling chamber costs US $ 100,000.

Still, cooling therapies are used for cooling the scalp to prevent hair loss (alopecia) from chemotherapy procedures. There are more than 14 million new cases of cancer recorded per year in the world, where chemotherapy can induce hair loss by up to 65% of the time, but scalp cooling systems are poorly marketed around the world, due to high cost of acquisition and still require their use in hospitals and follow-up by nurses and doctors.

Another application of cutaneous/muscular cooling is for aesthetic purposes. In the market there are specific masks for facial cooling that besides being used in analgesia treatments and recovery of lesions they are used for aesthetic procedures such as treatment of dark circles, pore closure and for absorption of specific products. However, most of these masks must be cooled prior to use and may result in the application of an inappropriate temperature for contact with the skin surface, causing burns and irritation.

In the search at the state of the art in scientific and patent literature, the following documents dealing with the subject have been found:

GAME READY Accelerated Recovery System (Game Ready Inc., Berkeley, Calif.) discloses a system in which it fills the console with a mixture of ice and water to prepare the console for operation and places a cooling pad in contact with an injured area of a patient or athlete. The user then activates the console which, in turn, circulates the cold water inside the console through the cooling block. Ice-cooled water reduces the pad temperature and reduces the site temperature of the patient's injury to minimize tissue swelling and damage. However, such device does not control the temperature, using only the liquid already cooled for the application, since its focus is related to the treatment of lesions and, therefore, makes it impossible its application to situations in which temperature control is necessary.

Cryomag Professional is a device that allows the synergistic use of a compression mechanism, with the thermal bag and magnetic therapy. However, such device uses separate parts to cool different parts of the body simultaneously. Such a factor making treatment expensive and cannot offer the same cooling rate for all the regions to be treated. Furthermore, the device is sold for the injuries treatment, and does not perform temperature control during the cooling, which makes it impossible its application to situations such as performance improvement with pre-workout cooling.

In the scalp cooling market for the prevention of CIA (chemotherapy-induced alopecia), there are two types of equipment acting in the sector; helmets with fluid circulation and helmets with cooled gel. The liquid system consists of a helmet connected to a cooling unit that circulates fluids through channels in the helmet. Those using gel helmets are actively cooled by high-powered freezers or dry ice refrigerators.

Paxman is an England-based company and sells the Paxman® Scalp Cooling System, and Digitana AB is a Sweden-based company and sells the DigniCap® Scalp Cooling System. Both are scalp cooling equipment for the prevention of hair loss during chemotherapy. The system consists of a silicone helmet attached to a fluid cooling unit. The coolant circulates through channels in the helmet. The machine must be switched on in advance to achieve the constant operating temperature before use, which takes approximately 40 minutes. Thereafter the pre-cooling of the scalp, which takes about 30 minutes, is performed and should be performed prior to the start of chemotherapy. Then the patient should use the equipment throughout the chemotherapy course and remain with it for a further 90 minutes after the end of the course. In this way, the treatment requires that the patient move to the place of application and that the patient remains in the armchair during the entire period of the course, implying a tedious and uncomfortable situation for the patient. In addition, they require use of the armchairs where the chemotherapy courses are performed. As a result, the time for armchair rotation is impaired, which reduces the number of chemotherapy courses performed at each center.

US 20140222121 A1 discloses an athletic heating/cooling system and method comprising thermal pads with thermal fluid channels and bending and stretching capability. System comprises fluid recirculating for temperature control. However, the system requires insertion of the fluid at the temperature at which the patient will be subjected in a container, such as ice or hot water, to then circulate the fluid in the device.

U.S. Pat. No. 7,896,910 relates to a modular device for therapy in an animate body comprising a heat transfer device geometrically adaptable to bodily regions of a patient. Said device requires prior cooling of the device fluid, which affects its efficiency over a long period of use.

U.S. Pat. No. 7,744,640B1 discloses a thermal treatment device with a gel layer with controlled temperature for treating specific parts of a patient's head, the temperature of the gel layer is controlled by an external thermal treatment layer. However, the device has a spiral gel contacting arrangement with the patient and, therefore, the gel absorbs a lot of heat and reduces the efficiency of the heat treatment, yet gel circulation in the device is hampered by its high density. Further, although the document implies the portability of the device, it does not mention a sufficient technical foundation to allow such portability of the device.

EP 2236048 A1 relates to a wearable cooling device which has a container of cooled water or ice for subsequent water circulation in the user's body. Therefore, the system requires the user to insert cooled water prior to its use, which also implies a lower efficiency in a long period of use.

EP1080648A2 relates to a wearable personal cooling system with fluid circulation. However, the fluid circulates in two envelopes, one front and one rear, such an arrangement has low efficiency and therefore the device would not apply in situations of injury recovery or medical treatment, only for the thermal comfort of a user.

Thus, from the literature researched, no documents were found anticipating or suggesting the teachings of the present invention, so that the solution herein proposed has novelty and inventive activity against the prior art.

Therefore, it is necessary to develop a more efficient body temperature control system that allows the temperature control of cutaneous and muscles surfaces by direct or indirect contact, allowing autonomy of operation, portability and lower acquisition cost.

### SUMMARY OF THE INVENTION

Thus, the present invention describes a system and method of temperature control for cutaneous and/or muscle surface by direct and/or indirect contact that provides improvement in recovery from injury, improvement in post-exercise muscle recovery and improvement in performance by regulating body temperature (e.g. delaying body hyper-heating), besides being able to be used in analgesia, to provide comfort to the user, in postoperative procedures, aesthetic procedures, prevention of alopecia, whether induced by chemotherapy or not, etc. The structure developed in the present invention provides a greater contact area with surfaces that will be target to temperature control and heat transfer, which provides better results achieved by users.

In a first object, the present invention provides a temperature control system for a cutaneous and/or muscular surface comprising at least:

a. one control unit;

b. one thermoelectric module;

c. one primary heat exchanger (6);

d. one pump; and

e. one secondary heat exchanger (1);

wherein,
control unit is connected to the thermoelectric module;
thermoelectric module is associated to the primary heat exchanger (6); and
pump is connected to the primary heat exchanger (6) and to the secondary heat exchanger (1).

In a second object, the present invention provides a method of temperature control for a cutaneous and/or muscular surface being implemented in the system as previously described, in addition to comprising at least the steps of:
a. adaptation of the contact area of the secondary heat exchanger (1) with the cutaneous and/or muscular surface;
b. definition of the fluid temperature;
c. current control in the thermoelectric module by the control unit;
d. pumping of circulating fluid in the primary heat exchanger (6) and secondary heat exchanger (1);
e. recirculation of the fluid of the secondary heat exchanger (1) to the primary exchanger (6);
   wherein, said method is implemented in a closed loop.

Furthermore, the inventive concept common to all claimed protection contexts is to provide a system and method that provides improved recovery from injury, improved post-exercise muscle recovery, hair loss prevention, analgesia, thermal comfort, post-operative treatment, performance improvement, and etc. by regulating body temperature, wherein said system and method relates to cooling or heating of the cutaneous and/or muscular surface by means of an arrangement, wherein a heat exchanger is arranged on said surface, wherein one control unit is responsible for controlling the temperature of the fluid circulating inside said exchanger by the actuation of a thermoelectric module.

These and other objects of the invention will be readily appreciated by those skilled in the art and by companies having interests in the segment, and will be described in sufficient detail for their reproduction in the following description.

### BRIEF DESCRIPTION OF THE FIGURES

In order to better define and clarify the content of the present application the following figures are presented:

FIG. 1 shows a frontal view of an embodiment of the system of the present invention wherein the secondary heat exchanger (1) is adapted to be associated with a user's scalp.

FIG. 2 shows a back view of the embodiment described in FIG. 1.

FIG. 3 shows a side view of the embodiment described in FIG. 1.

FIG. 4 shows the construction of the embodiment of FIG. 1 in detail.

FIG. 5 shows a preferred example of the embodiment of FIG. 1 further comprising an adaptation structure (4.2), an insulation layer (4.1) between the fluid inlet tube (2.1). It further presents a support structure (5) for better positioning and fixation of the elements during use by the user.

FIG. 6 shows a back view of the preferred example described in FIG. 5.

FIG. 7 shows the detailed support structure (5) mentioned in the description of FIG. 5.

FIG. 8 shows details of system embodiment of the present invention wherein the secondary heat exchanger (1) is adapted to be associated with a user's facial region.

FIG. 9 illustrates details of a system embodiment of the present invention wherein the secondary heat exchanger (1) is adapted to be associated with the lower muscle group of a user.

FIG. 10 illustrates a frontal view of the arrangement of the secondary heat exchanger (1) described in FIG. 9 in the lower muscular region of a user.

FIG. 11 illustrates a rear view of the arrangement of the secondary heat exchanger (1) described in FIG. 10 in the lower muscular region of a user.

FIG. 12 illustrates the detailed arrangement of the secondary heat exchanger (1) adapted for association in the lower muscular region of a user.

FIG. 13 illustrates the detailed arrangement of the secondary heat exchanger (1) adapted for association in the lower muscular region of a user, also detailing the fluid inlet (2) and outlet (3) means.

FIG. 14 illustrates the detailed arrangement of the secondary heat exchanger (1) adapted for association in the lower muscular region of a user, more specifically in the calf region of a user.

FIG. 15 illustrates the detailed arrangement of the secondary heat exchanger (1) adapted for association in the upper muscular region of a user, more specifically in the dorsal region.

FIG. 16 illustrates the detailed arrangement of the secondary heat exchanger (1) adapted for association in the upper muscular region of a user, more specifically in the region of the forearm.

FIG. 17 shows an embodiment of the system in a situation where control unit, thermoelectric module, primary heat exchanger (6) the pump are arranged in a portable module.

FIG. 18 shows a rear view of the embodiment of FIG. 17.

FIG. 19 shows a frontal view of the embodiment of FIG. 17.

FIG. 20 shows an embodiment in which the portable module described in FIGS. 17 to 19 is used in conjunction with secondary heat exchanger (1) arranged as described in FIGS. 9 to 13.

FIG. 21 refers to a rear view of the embodiment of FIG. 21.

FIG. 22 relates to a construction embodiment of the thermoelectric module in an upper view.

FIG. 23 relates to a construction embodiment of the thermoelectric module in a rear view.

FIG. 24 shows a detailed embodiment of the secondary heat exchanger (1) comprising an internal layer (1.1) and an external layer (1.2).

FIG. 25 shows a detailed schematic of the embodiment of the thermoelectric module.

FIG. 26 shows a detailed embodiment of the secondary heat exchanger (1) adapted for association in the upper muscular region of a user, more specifically in the forearm region.

FIG. 28 refers to a block diagram indicating the operation of the temperature control system for a cutaneous and/or muscular surface.

FIG. 29 discloses an embodiment of the secondary heat exchanger (1) comprising the arrangements of the layers in hive configuration.

### DETAILED DESCRIPTION OF THE INVENTION

The following descriptions are presented by way of example and not limiting the scope of the invention and will make the object of the present patent application more clearly understood.

In a first object, the present invention provides a system for temperature control for a cutaneous and/or muscular surface, wherein said system comprises at least: a control unit; a thermoelectric module; a primary heat exchanger (6); a pump; and a secondary heat exchanger (1). The present system basically comprises the control unit connected to the thermoelectric module, where the thermoelectric module, in turn, is associated with the primary heat exchanger (6) and also a pump is connected to the primary heat exchanger (6) and to the secondary heat exchanger (1).

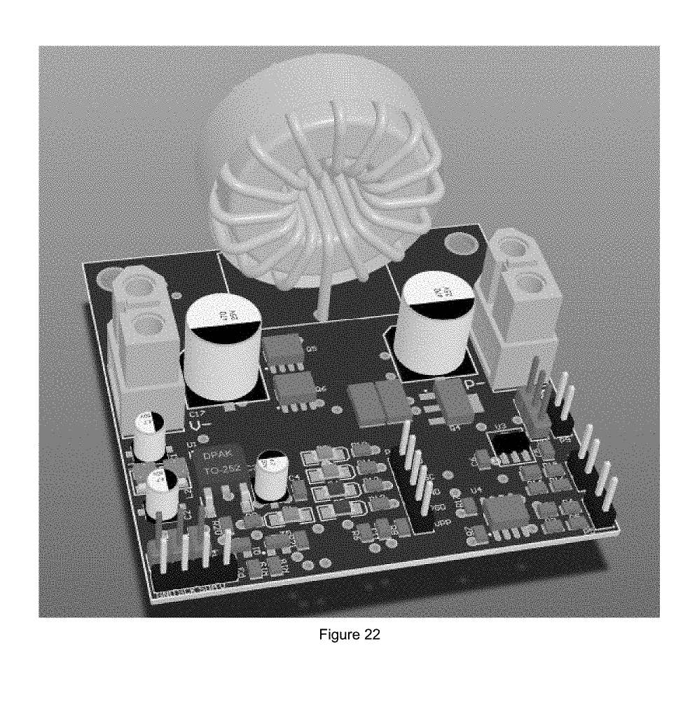

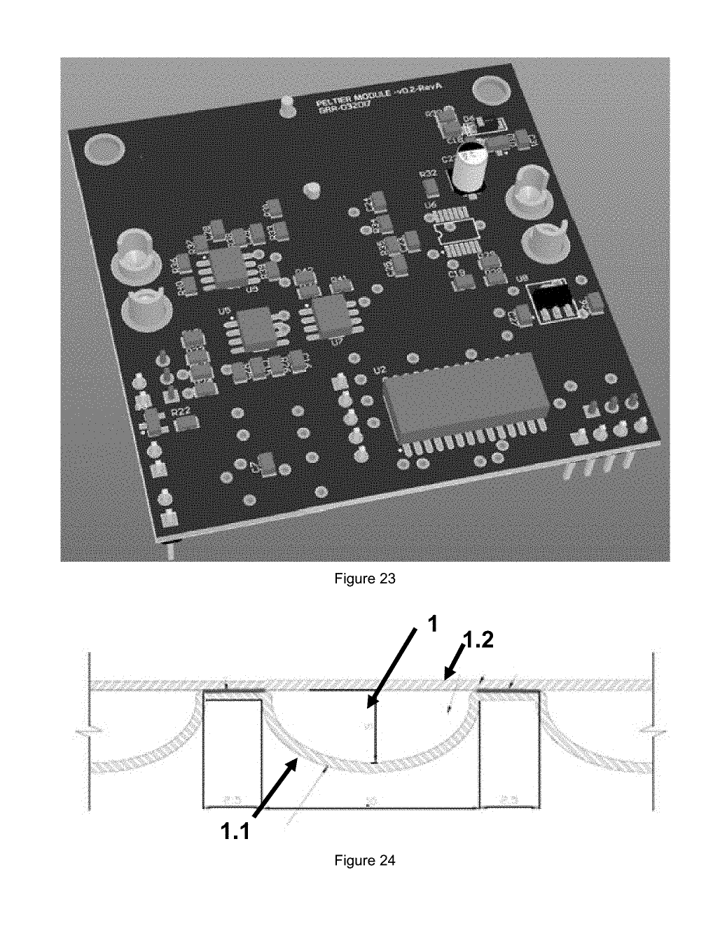

The system control unit is any device capable of being configured to perform temperature control of peripherals thereof associated where this device emits and receives control signals and, through these signals, communicates with said peripherals. Furthermore, this control unit can be electronic and programmable, where the programming can reach only the programmer level (i.e. only the programmer has programming permission) or reach the user level, so that said user can be able to configure the device, and in this last example, the user can set which temperature to use the system. In one embodiment, the control unit is a microcontroller. In a preferred embodiment, the control unit is a microcontroller associated with input and output peripherals that allow interaction with a user, in addition to providing other utilities for better operation, for example the use of LEDs, I/O buttons, voltage regulators, capacitors, resistors, power transformers and other electronic components. An embodiment of said control unit is shown in FIGS. 22 and 23.

Further, the control unit comprises at least one electric current controller, where this controller can be implemented by physical means, i.e. by electronic components arranged to perform such function, or by means of the microcontroller itself, which is programmed via software, i.e., by code lines, to control the electric current at the output of its terminal and/or the electric current which is directed to some of the peripherals. In one embodiment, the current controller was implemented via software. In addition, this electric current controller is responsible for temperature control of the thermoelectric module. Also, the current controller is able to define the intensity and direction of the electric current being driven to the thermoelectric module.

The thermoelectric module, in turn, is any device capable of performing heat exchange by applying electric current, wherein said module can be composed of thermally conductive plates, which allow increase the efficiency of the heat exchange. Also, the heat exchange produced by the thermoelectric module is relative to the electric current applied at its terminals, either by intensity or polarity. In one embodiment, the thermoelectric module comprises heat sink or heat dissipation surface, i.e., the same surface is capable of absorbing heat from an environment or element and dissipating heat in an environment or element where such surface capacity is defined by the electric current applied to the module electrical terminals. For exemplifying purposes, when the electric current is applied in a given direction the surface absorbs heat from an environment and, when the polarity is reversed, the electric current passes through in an inverse direction, and thus the surface is capable of dissipate heat. In one embodiment, the thermoelectric module is composed of two surfaces, while one absorbs heat, the other surface dissipates heat. In one embodiment, the thermoelectric module may be a Peltier effect tablet.

Thus, the thermoelectric module is associated with the primary heat exchanger (6), where this association is thermal, that is, these elements are associated to perform heat exchange with each other. Also, more specifically, the surface of the thermoelectric module is connected to the primary exchanger (6), so that this connection is physical, in the intention of providing better efficiency. As previously mentioned, this surface is capable of absorbing heat or dissipating heat, so this thermal association allows the thermoelectric module to be responsible for absorbing heat or dissipating heat in said primary heat exchanger (6).

The primary heat exchanger (6) is any element capable of performing thermal exchanges, allowing a fluid circulation, either in its interior or on its surface. In the present system, the primary heat exchanger (6) is driven by a fluid which is heated or cooled by the thermoelectric module, as described above. Furthermore, the primary heat exchanger (6) is connected to the secondary heat exchanger (1), where both are fluidly connecting. This association between the heat exchangers is due to the primary heat exchanger (6) is responsible for balancing the temperature of the fluid going to the secondary heat exchanger (1), since the primary exchanger (6) is associated with thermoelectric module and the heat from the module is transferred to the fluid by means of said primary exchanger (6). This type of arrangement has been developed in order to allow the fluid to be approximately in the temperature desired by the user, since when the fluid flows through the region in contact with the user, it tends to lose/gain heat (depending on the application), unbalancing the temperature set by the user.

Nevertheless, this association is not necessarily physical, that is, it does not refer to having two individual heat exchangers connected with each other. The primary (6) and secondary (1) heat exchanger description of the present invention refers to the fact that the primary exchanger (6) is in contact with the thermoelectric module and the secondary heat exchanger (1) is in contact with the user, where the terms “primary” and “secondary” refer only to which element the heat exchanger performs the heat exchange. That is, it can be understood that the present system may use two heat exchangers or only one, in the case of a single exchanger, it is provided with a primary region (6) in contact with the thermoelectric module and a secondary region (1) in contact with the user.

In addition, in order for the fluid to flow between the primary heat exchanger (6) and the secondary heat exchanger (1), the present system comprises a pump associated with the exchanger. The pump may be positioned in any region of the circuit representing the system, e.g., being positioned between the primary (6) and the secondary (1) exchangers, so that the pump is connected at the outlet of the primary exchanger (6) and at the inlet of the secondary exchanger (1). In another example, the pump may be connected at the outlet of the secondary exchanger (1) and at the inlet of the primary exchanger (6). In view of the above, in cases where the exchanger is a single element, the pump is connected to the inlet and outlet of said exchanger, the inlet being close to the primary region (6) and the outlet is close to the secondary region (1). Finally, the pump can be of any conventionally used model, for example, piston pumps, centrifugal pump, gear pump, vane pump, positive displacement pump, diaphragmatic pump, etc.

This arrangement allows the system to operate in a closed loop, i.e. so that the fluid leaving the secondary heat exchanger (1) is returned to the primary heat exchanger (6), avoiding the need to use a large container for the fluid, since it is only available in the heat exchangers, being able to be circulating or static.

Further, the system needs to ensure that the temperature of the reaching secondary heat exchanger (1) is at a user-set temperature. For this, the control unit may be configured to apply a certain electric current to the thermoelectric module, ensuring that the module operates at a certain temperature. Furthermore, in one embodiment, the system comprises a temperature sensor which is associated with the control unit, allowing the closed loop operation, where this sensor acts as a feedback to the unit, and thus the control unit can adjust the current controller to provide the electrical current needed to maintain the desired temperature, since the user may be varying the temperature of his body, for example, in the practice of physical exercises. Hence it may be necessary to vary the temperature of the thermoelectric module to ensure that the temperature in the exchanger is in the vicinity of the desired temperature.

Said temperature sensor may be positioned so as to measure: the fluid temperature within the heat exchanger; the temperature on the surface of the heat exchanger; or the user's cutaneous and/or muscular surface temperature; where in the last one the best result is guaranteed, since the desirable, depending on the application, is to control the temperature directly from said cutaneous and/or muscular surface.

For exemplifying purposes, the fluid used may be any type of fluid having sufficient properties for heat transfer. For example, the fluid may be a gas, water, antifreeze fluid, ethylene glycol, propylene glycol, vaseline, among others.

The system of the present invention further comprises an independent mains power supply associated with the control unit and the pump, where this power supply is capable of providing sufficient electrical power for the operation of these elements. Said power supply may be any type of independent generator, such as rechargeable or non-rechargeable batteries. In one embodiment, a lithium ion battery was used. Moreover, the system is adapted to also operate with the use of the electric grid itself. Thereby, the system comprises a selector switch device that allows define which power supply supplies power to the system, and this selection is defined as the use. In one embodiment, this selector switch performs automatic switching between power supplies. In one embodiment, the selector switch automatically switches to use with the electric grid when the user connects the system to the electric grid itself and, when the user disconnects from the electric grid, the selector switch automatically switches to the use of the independent power supply, in this case, a battery. Also, when the system is connected to the electric grid, the battery is recharged.

This type of arrangement was developed to allow the system to be portable, allowing the user to load the system into any environment and use it for whatever application. Thus, in case the user wishes to charge the equipment for, for example, its use during physical exercises, he just has to use the battery.

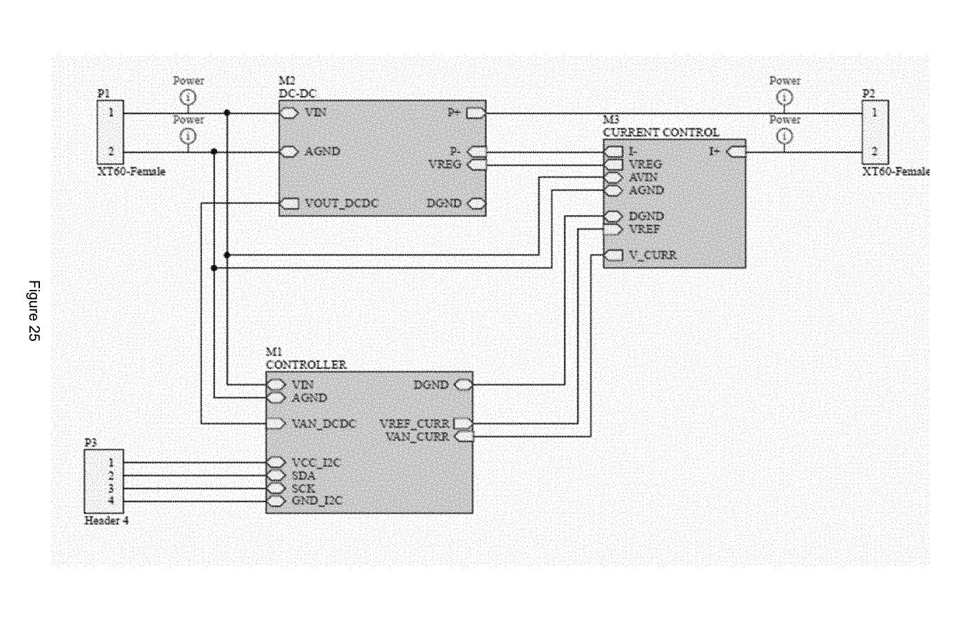

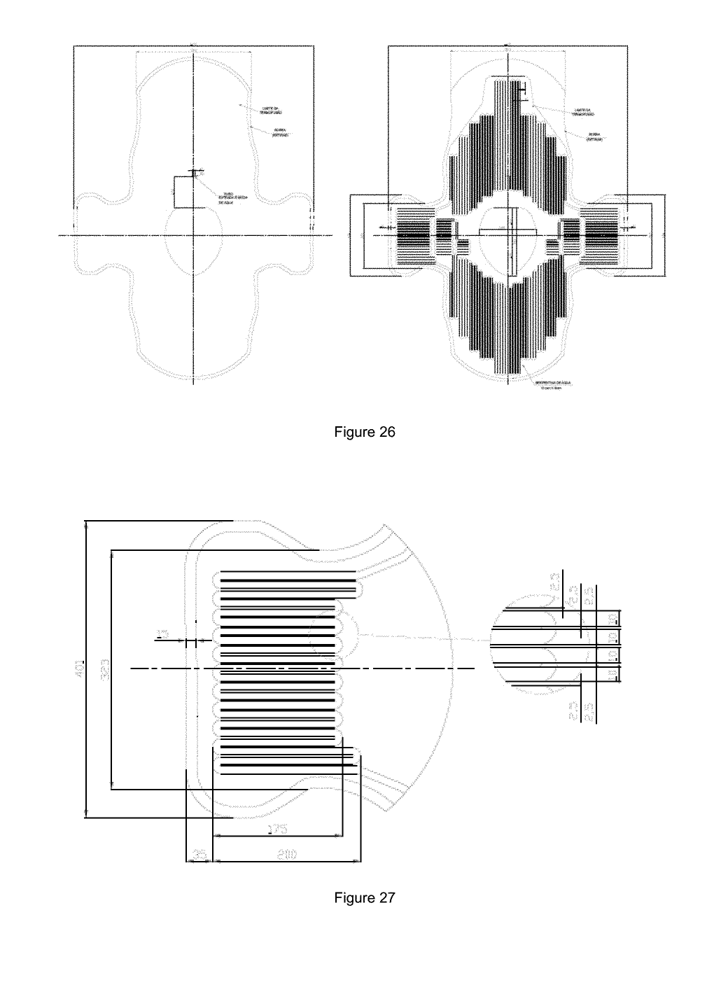

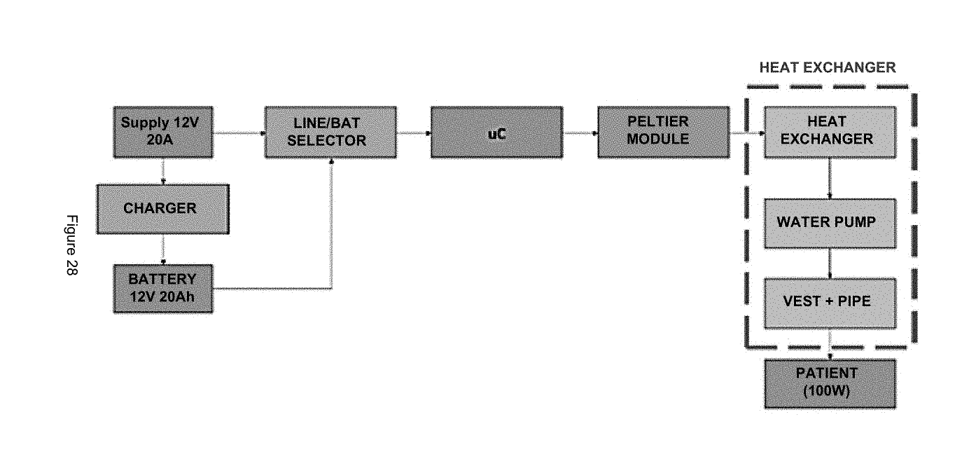

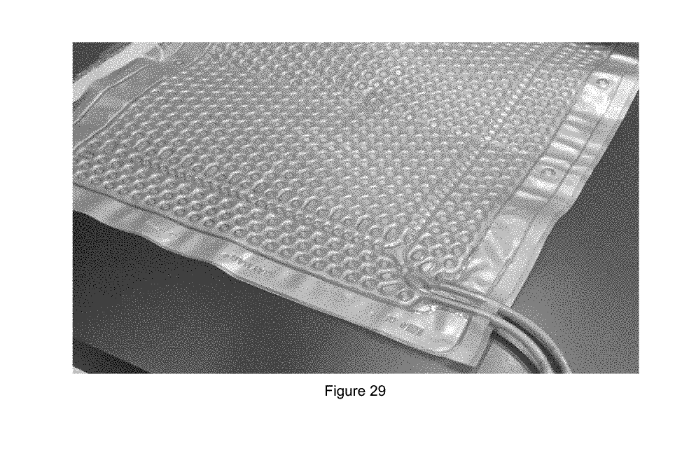

In turn, the secondary heat exchanger 1, which connecting with the primary heat exchanger (6), comprises at least one internal layer (1.1) and an external layer (1.2) so that the internal layer (1.1) is the one responsible for entering into contact with the cutaneous and/or muscular surface, either directly, when the layer directly touches the region of the user, or indirectly, when the layer is enveloped or accompanied by a fabric. In one embodiment, the internal layer (1.1) is formed by a cavity in its cross section and the external layer (1.2), in its cross section has a flat geometry, so that when the external layer (1.2) overlies said internal layer (1.1), a channel is formed for the passage of the fluid. Such an arrangement can be seen in FIG. 24.

The internal layer (1.1) of the secondary heat exchanger (1) is provided with thermal conductive characteristics, these characteristics being of the nature of the material itself or some other type of thermal coating which generates such functionality. By thermal conductive characteristics is meant, in the present invention, any composition which allows high efficiency in the exchange of heat between the fluid and the skin and/or muscular surface of a user.

Furthermore, the internal layer (1.1) comprises a comprehensive contact area relative to the cutaneous and/or muscular surface, wherein by wide contact area is meant any geometry capable of providing greater contact area of the layer with the cutaneous and/or muscular surface of the user, which is desired to control the temperature. Thus, the geometry of the internal layer (1.1) is defined according to the application with which the system is being used, wherein the external layer (1.2) needs to have the same geometry so that overlap between the layers is possible, forming the exchanger secondary (1). Thus, as a whole, the secondary heat exchanger (1) is arranged to achieve greater efficiency in temperature control also due to the greater contact obtained with the user's cutaneous and/or muscular surface. For exemplifying purposes, the secondary heat exchanger (1), and its layers, may comprise geometry adapted to lower limbs, upper limbs, chest, scalp region, facial region, etc., being for animals or humans.

The external layer (1.2), in turn, is arranged so as to decrease the heat exchange between an external medium and the fluid inside the secondary heat exchanger (1). For this purpose, the external layer is disposed in contact with an outer surface, wherein said outer surface can be defined by: any external environment, in which embodiment the external layer (1.2) is effecting heat exchange directly with the external environment, wherein arrangement is found to be less efficient for the system, but can be used in cases where greater efficiency is needed; a thermal insulator layer, wherein the external layer (1.2) is coated by a layer of thermal insulation, drastically reducing heat exchange with the external environment; or a heat sink, wherein said sink can be composed of fins or, by heat sink, is meant any structure capable of dissipating heat, regardless of its geometry.

However, the external layer (1.2) may contain thermal insulation characteristics as property of the material itself to which said outer layer (1.2) is formed. Thereby, the external layer (1.2), in one embodiment, is formed of thermal insulator material, reducing the heat exchange with the external environment to which it is associated.

Thus, based on the above, the secondary heat exchanger (1) can be defined as being a serpentine, wherein the layer arrangements are based on the same characteristics defined above; hive; or any other tubular element capable of allowing fluid circulation therein.

In a second object, the present invention provides a method of temperature control for a cutaneous and/or muscular surface, this method being implemented in the system detailed above, so that the present method comprises at least the steps of: adaptation of contact area of the secondary heat exchanger (1) with the cutaneous and/or muscular surface; definition of fluid temperature; current control in the thermoelectric module by the control unit; pumping of circulating fluid in the primary heat exchanger (6) and secondary heat exchanger (1); and recirculation of the fluid from the secondary heat exchanger (1) to the primary exchanger (6). Furthermore, the method is implemented in closed loop, i.e., as mentioned in the last step, the fluid is recirculated from the secondary heat exchanger (1) to the primary heat exchanger (6).

First, in the step of adapting the contact area of the secondary heat exchanger with the cutaneous and/or muscular surface occurs in the geometric preparation of said secondary heat exchanger (1). Thus, it is defined in which region of the user the method is applied, and therefore the exchanger is made or selected among existing ones, and disposed over the user's region. In one of the numerous examples, it was defined that the muscular group of the lower limbs is the region to be submitted to the temperature control; therefore, the secondary heat exchanger (1) is adapted to involve the lower limbs of the user.

In sequence, as an additional step, the exposure time in which the skin and/or muscular surface is subjected to the secondary heat exchanger (1) is defined. Thus, the temperature of fluid circulating in the secondary heat exchanger (1) is defined. These steps are defined to avoid some discomfort for the user, preventing the user member from being exposed to a very high or very low temperature for a very long time.

As soon as the temperature is set, the control unit applies an electric current, through the current controller, to the thermoelectric module, whereby it is possible to define which temperature the thermoelectric module operates on the primary heat exchanger (6). Thereby, the circulating fluid is pumped between the primary (6) and the secondary (1) exchangers, where if the thermoelectric module is set to heat the fluid in the primary heat exchanger (6), the heated fluid is circulated to the secondary exchanger (1), and in case of cooling of the fluid, the cooled fluid is pumped to the secondary exchanger (1). Finally, since the system operates in a closed loop circuit, the fluid exiting from the secondary heat exchanger (1) is recirculated to the primary exchanger (6).

In addition, as an additional step, the temperature measurement is performed so that a closed loop control is performed, in order the measurement can be made within the heat exchanger; on the surface of the heat exchanger; or on the user's own cutaneous and/or muscle surface. This type of operation allows a better temperature control to be obtained, since the control unit is able to almost detect the temperature reached with the steps performed by the method of the present invention, thus regulating the current control as required.

This method further allows the user to choose how to use the developed system so that it can be used connected to both a mains-dependent power supply and a mains-independent power supply, e.g., a battery or any voltage generator, since the system allows performing a switch between the power supplies. Therefore, the system is portable and can be driven to any place the user wishes.

### EXAMPLES

The examples shown herein are for the sole purpose of exemplifying one of a number of ways of performing the invention, but not limited to, the scope thereof.

### Example I

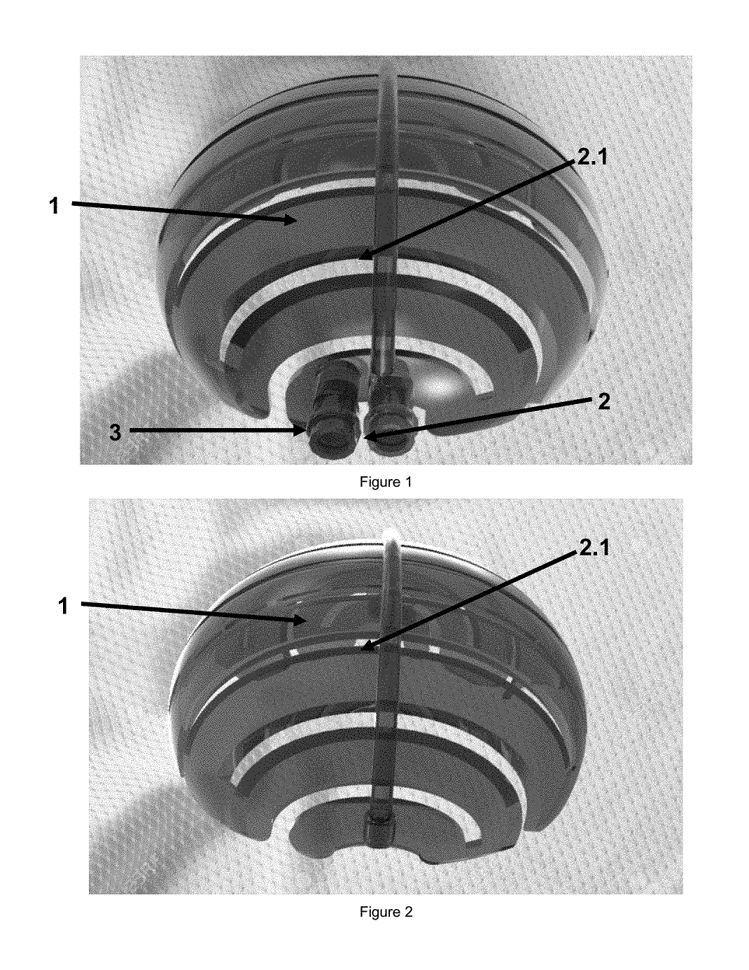

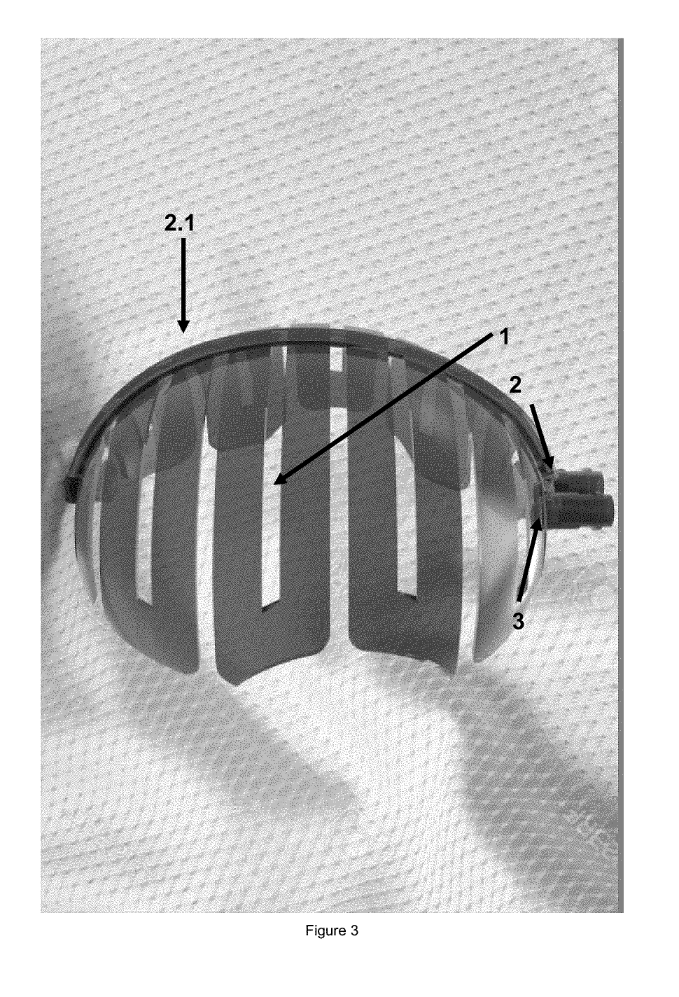

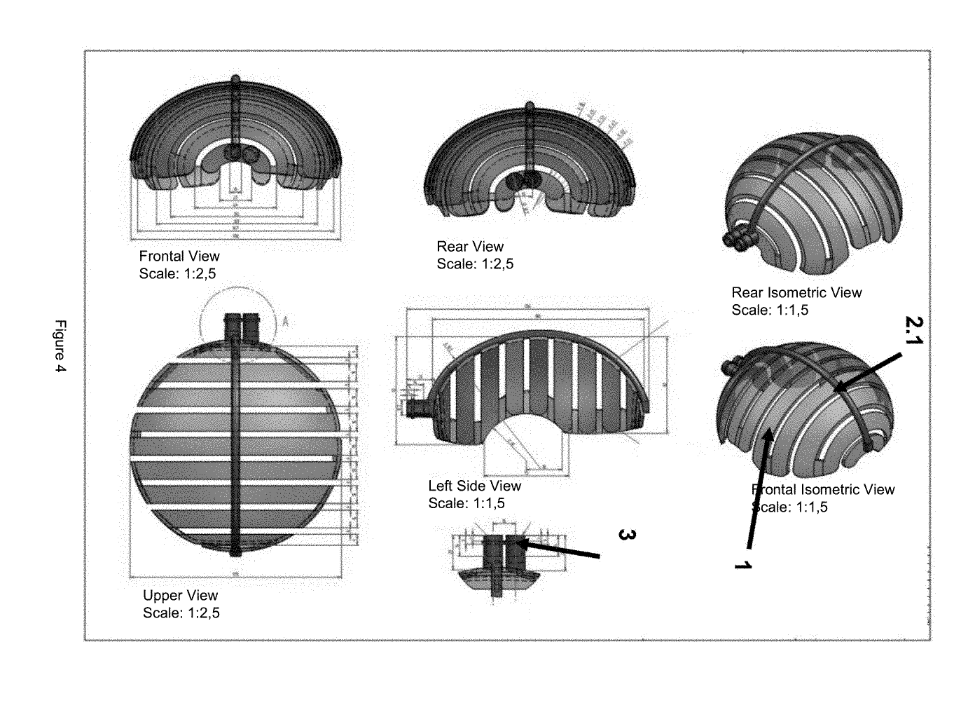

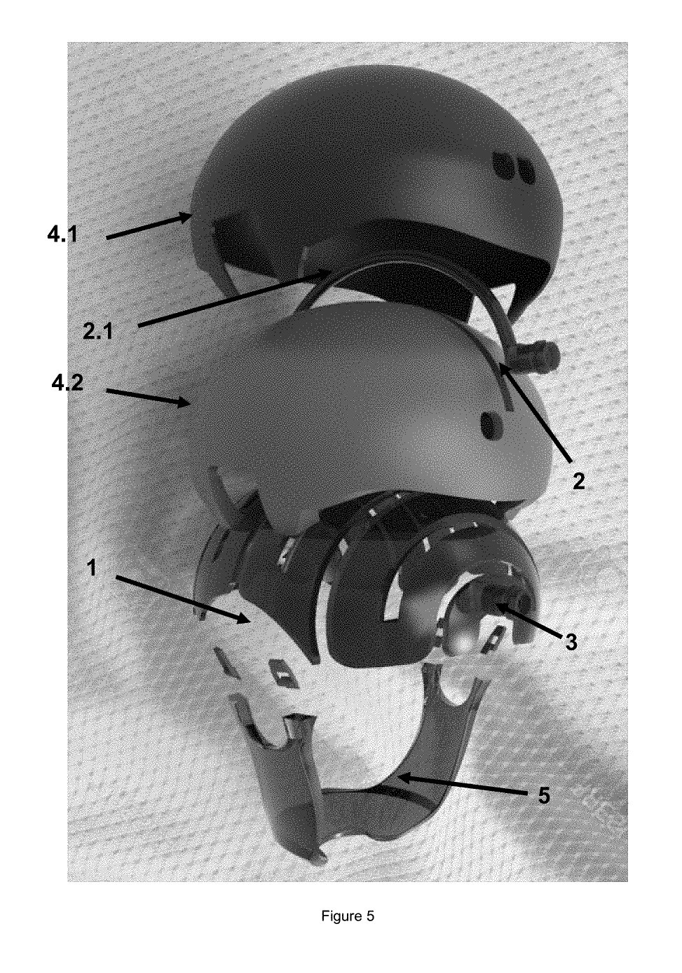

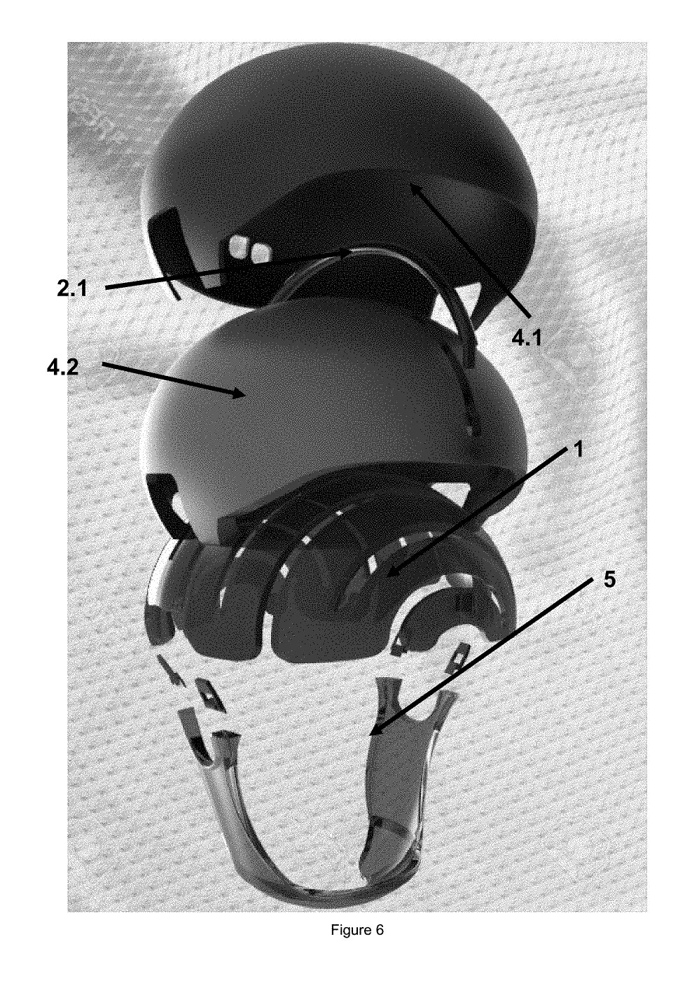

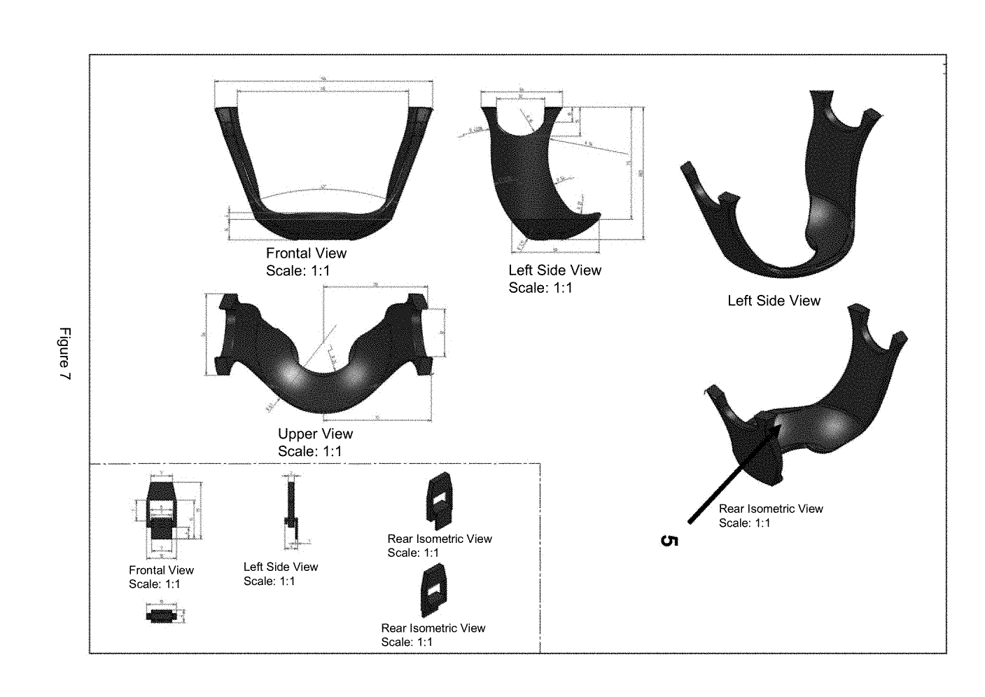

In a preferred example, the secondary heat exchanger (1) is adapted for association in a user scalp region as illustrated in FIGS. 1 to 7. Such construction is useful in situations for recovery from injury, analgesia, and thermal comfort of the head region. And, preferably, it is used for hair loss treatment. More specifically, the present arrangement is of great utility in the treatment of chemotherapy-induced alopecia (CIA).

In this situation, the secondary heat exchanger (1) is constructed in serpentine in order to optimize the contact surface with the user scalp, and therefore presenting an efficient heat exchange.

In addition, an insulation layer (4.1) and an adaptation structure (4.2) may be associated together with the primary heat exchanger (1). The adaptation structure (4.2) may be constituted of some insulating material.

Thus, when thermally isolating the fluid inlet tube (2.1) from the secondary heat exchanger (1), the efficiency of the device is improved, since the already cooled fluid does not lose or reduce heat loss upon contact with the secondary heat exchanger (1). Also, if the system is used to heat the area, the insulation prevents the fluid from losing heat to the environment or the secondary heat exchanger (1).

The adaptation surface (4.2) allows for a better geometric arrangement of the fluid inlet (2) and outlet (3), facilitating the portability and operation of the system.

In one embodiment, the secondary heat exchanger (1) has the external outer layer (1.2) of thermal insulation material, and thus, the fluid inlet tube (2.1) is also thermally insulated from the secondary heat exchanger 1.

Moreover, for this situation, a support (5) is associated with the secondary heat exchanger (1) in order to secure the contact of the exchanger (1) with the user skin region and to optimize the heat exchange. The support (5) also allows for greater comfort and stability for the user during heat exchange, again facilitating portability and ease of use of the device.

Therefore, the present example promotes a high efficiency of heat exchange, comfort to the user and allows portability and greater ease of use. Such advantages are of great importance in the treatment chemotherapy-induced alopecia since it would allow the user to move during treatment, a low cost of acquisition and a facility of operation that can be done by the user, presenting autonomy in its use.

### Example II

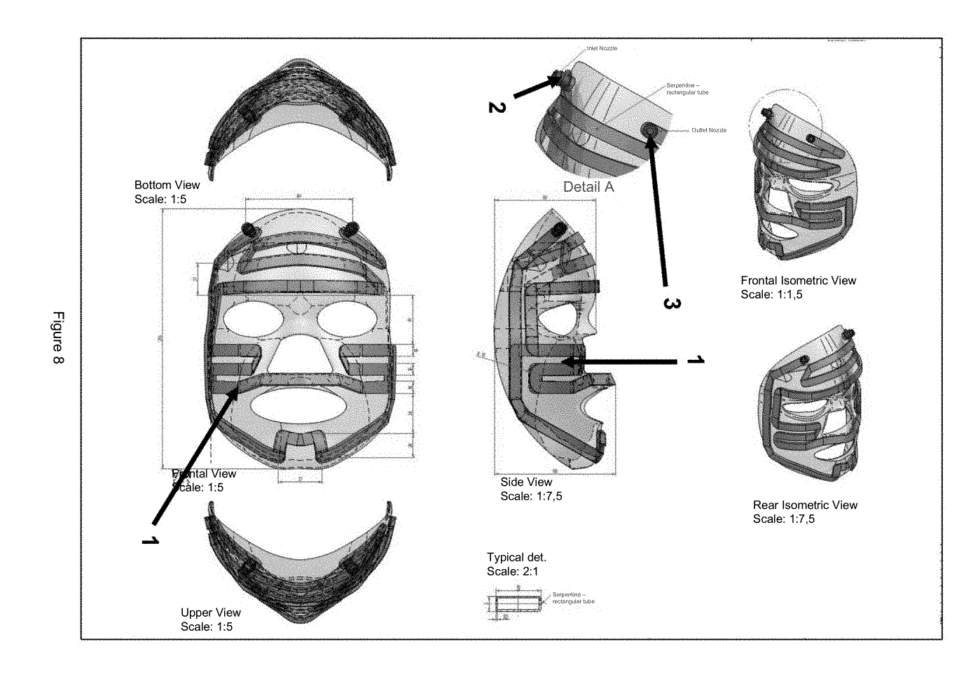

In one embodiment, the secondary heat exchanger (1) is adapted for association in the facial region of a user. FIG. 8 illustrates an example of said embodiment.

This embodiment can be used for the treatment of facial traumas and injuries, post-surgical recovery and post-aesthetic procedures, dark circles treatment, pore closure, dental, otolaryngologic, mandibular preoperative analgesia and other applications. In addition, it can be used to warm the facial area, opening pores, improving area blood circulation, among other applications.

Thus, the secondary heat exchanger (1) can be arranged in order to meet the need for application. For example, for an application in which the aim is the treatment of dark circles and swelling in the ocular region, the secondary heat exchanger (1) is constructed so as to encompass and optimize the surface to be treated. In an application for post-operative dental recovery, such as surgical extraction of wisdom tooth, the exchanger (1) is constructed in order to exchange heat with the buccal and mandibular region, thus making the recovery of the patient faster.

The system may include a textile layer for direct contact protection between the heat exchanger (1) and the subject skin surface, avoiding any possible discomfort to the patient.

Therefore, the present system allows the thermal treatment of the facial region with affordable cost, providing comfort and practicality to the user. In addition to providing an easy and effective way of analgesia and recovery offered by aesthetic clinics and healthcare professionals to its clients.

### Example III

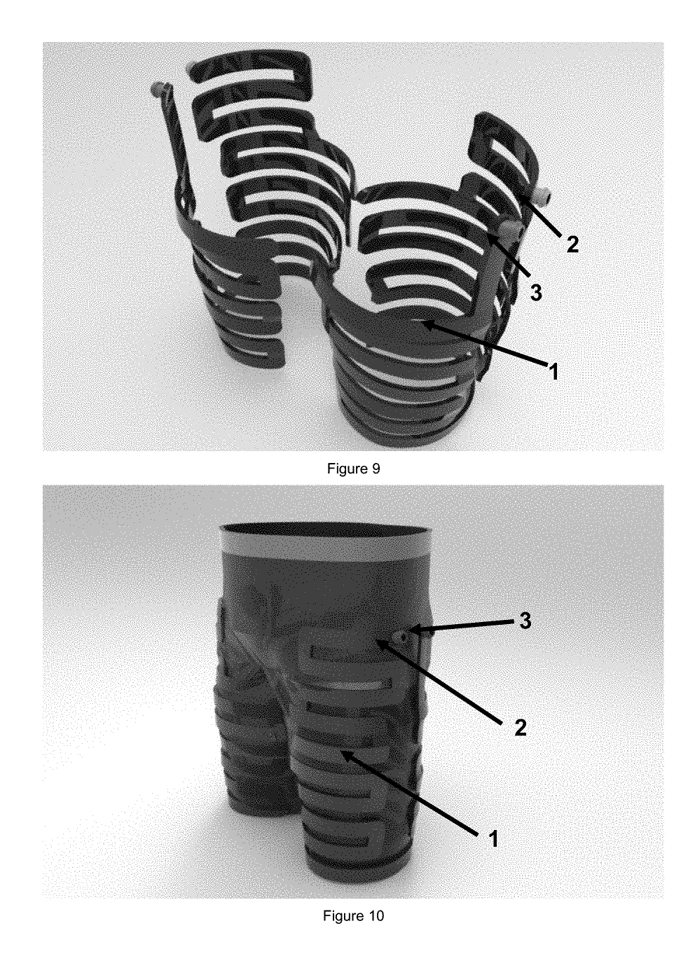

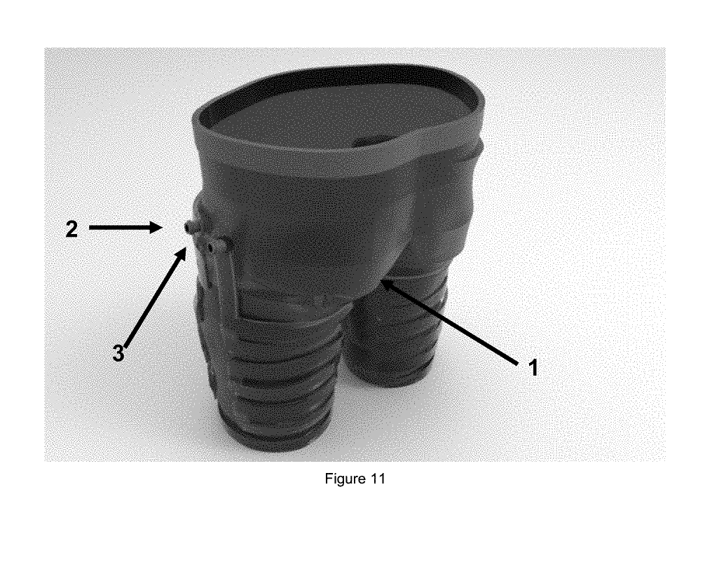

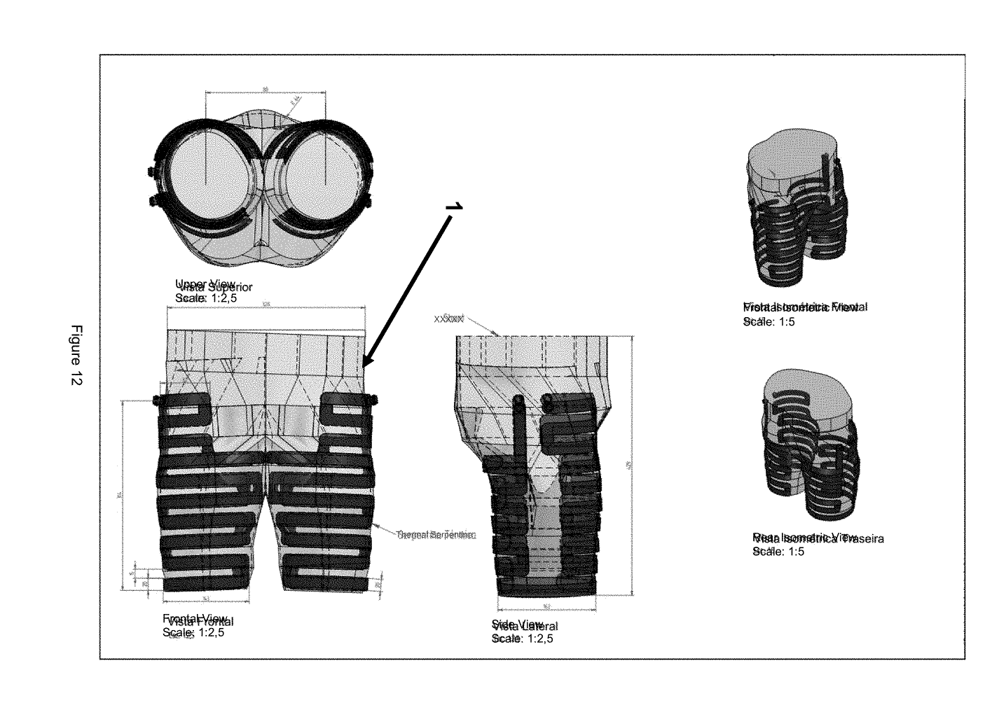

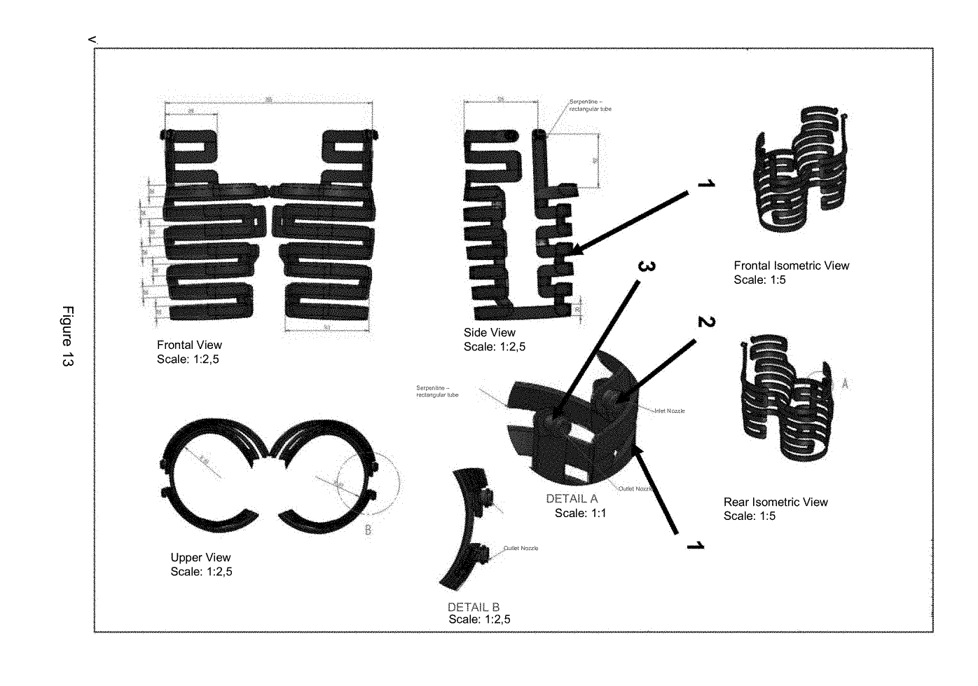

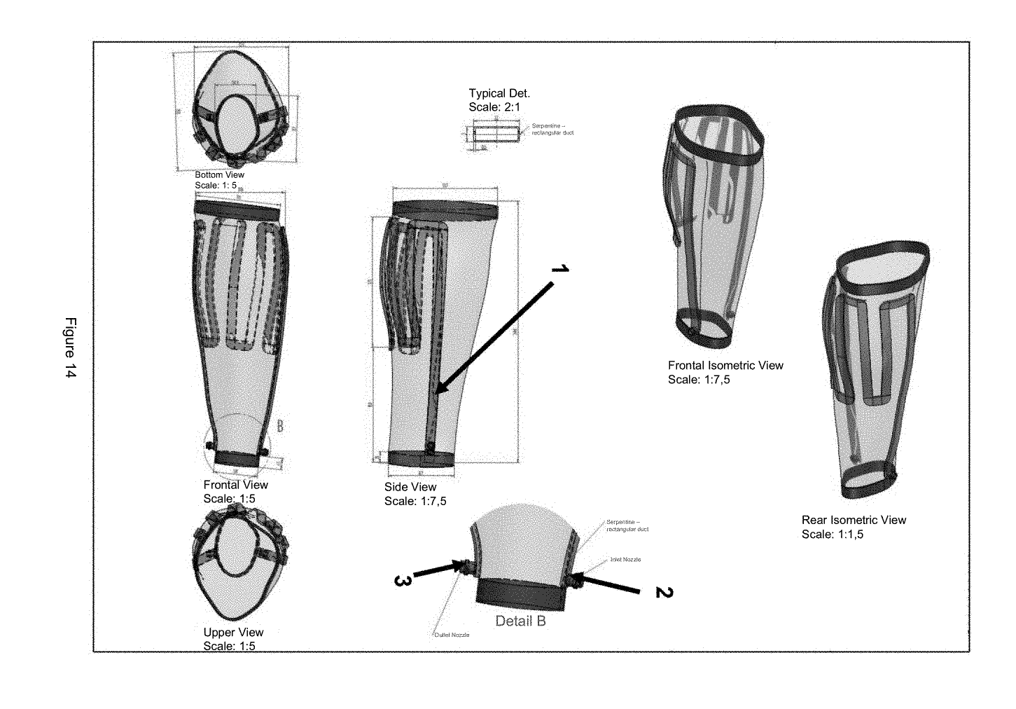

FIGS. 9 to 14 show an illustration of the temperature control device which is in contact with the lower limbs. In this situation, the secondary heat exchanger (1) is adapted to exchange heat with regions of the lower limbs such as legs, calves, feet, pelvic girdle and thighs.

In one application, the heat exchanger (1) encompasses the thigh and waist region as shown in FIGS. 9 to 13. In this situation, preferably the heat exchanger does not reach the perineal area and buttocks in order to promote greater comfort to the user during use.

In the application according to FIG. 14, the heat exchanger (1) encompasses the calf area, and the fluid circulates in the heating/cooling region as the user requirements.

Therefore, this configuration can be used for recovery from injuries, analgesia (for example, by contusion and in a postoperative situation), muscle recovery after or during training/practice of physical activities.

For example, a subject who engages in sports/physical activity may use the system for muscle cooling prior to performing the activity in order to increase its performance or also during physical activity to control his temperature during activity since the present system can be used in a portable way. After the activity, the subject can still use the system for muscle recovery and injury treatment.

A surfer, during the training or a competition in which he is subjected to a low temperature, can use the device in the lower limbs to keep them warm.

A researcher working in areas of glaciers or professionals subjected to very low temperatures can use the system to warm the lower limbs during the execution of their activities, avoiding situations of hypothermia or extreme cold.

On the other hand, professionals subjected to very high temperatures, such as restaurant staff working in kitchens, professionals working in deserts, among other applications can use the system to keep their lower limbs cooled and therefore improve their performance during the execution of the service.

In addition, the secondary heat exchanger (1) may be associated with immobilizers and accelerate the muscle recovery of subjects who have suffered fractures, ligament rupture and strong injuries.

Thus, the present system allows users to use a portable cooling/heating system, with high efficiency in the heat exchange and at an affordable cost.

### Example IV

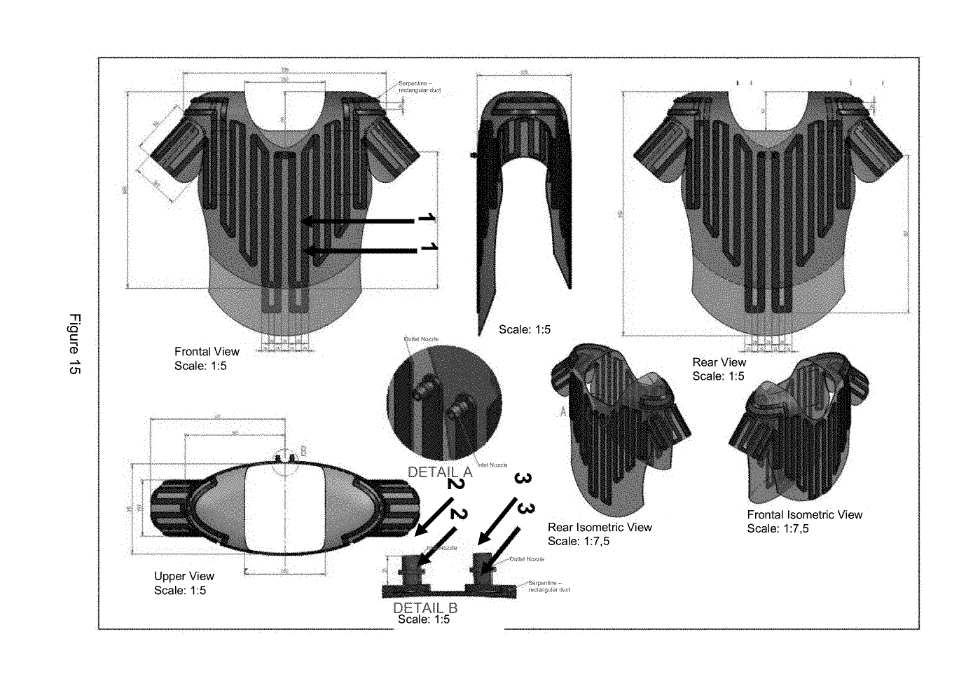

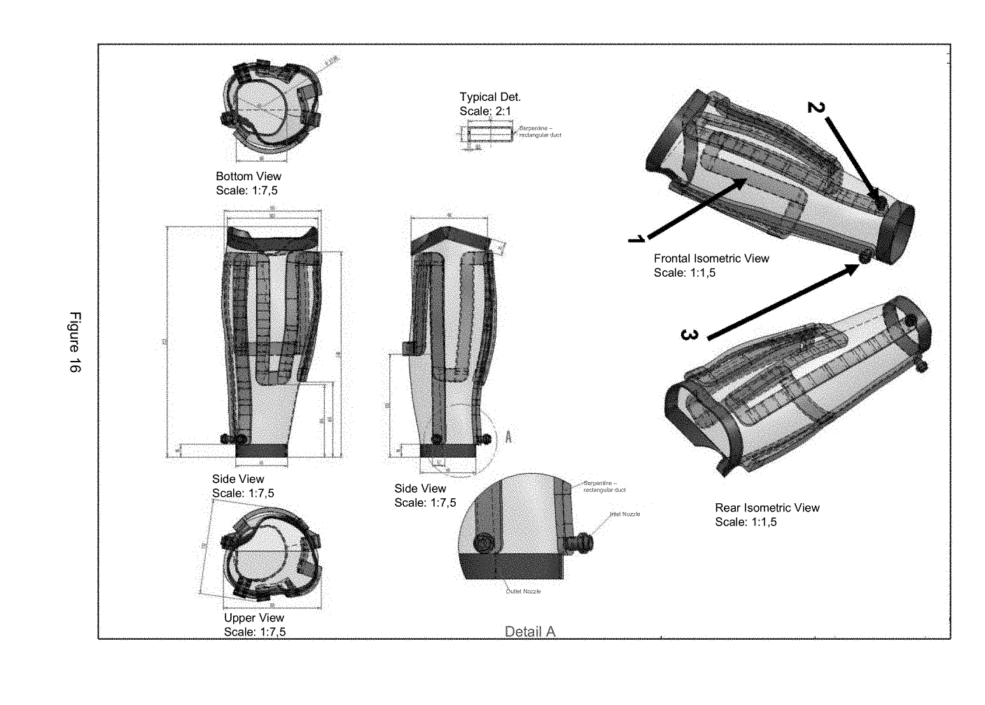

FIGS. 15 and 16 show embodiments of the secondary heat exchanger (1) adapted for associations to a user's upper limbs groups.

FIG. 15 shows an embodiment, where it is possible to heat/cool the dorsal region and shoulders of a user. Such a configuration may be applied, for example, in a combat contest situation. After the first round, the athlete positions the system in the dorsal region and/or shoulders and cools the region quickly, making recovery of injuries faster and increasing thermal comfort.

FIG. 16 shows an embodiment in which the heat exchanger (1) is used for cooling/heating the forearm area. Such an application is of great use in applications of recovery from injury and fracture, making recovery of the individual faster and providing greater thermal comfort.

In addition, this embodiment can also be used for the treatment of upper area traumas and injuries, post-surgical recovery, analgesia, among other applications.

### Example V

In a certain situation, the embodiments mentioned in examples I to IV can be combined in order to better serve the user's needs.

For example, an athlete may use cooling of the scalp, lower and upper limbs region in order to recovering lesions in various areas or muscular cooling of regions during training for thermal comfort or performance improvement.

A surfer can use the system in the dorsal region for cooling due to solar heating, and in the region of the lower limbs for heating the region due to the low water temperatures.

Additionally, a user who has suffered multiple injuries due to a major accident or high exposure situation can use the whole body system in order to make recovery of the lesions suffered faster.

A subject who has suffered an accident in a fire and who has severe burns may use the system covering the affected regions to provide greater thermal comfort during the recovery.

### Example VI

Further, the secondary heat exchanger (1) can be adjusted for application in other animals, such as horses, domestic pets, ruminants and so on.

Thus, the system can be used to recover lesions of the animals, analgesia, surgical procedures and etc. But it can also be used to increase the animal's performance while performing some task, such as transporting people, food and objects.

Moreover, the present system can be used in animals to improve their performance in competitions such as horse riding, racing, rodeo, sled racing and etc.

### Example VII

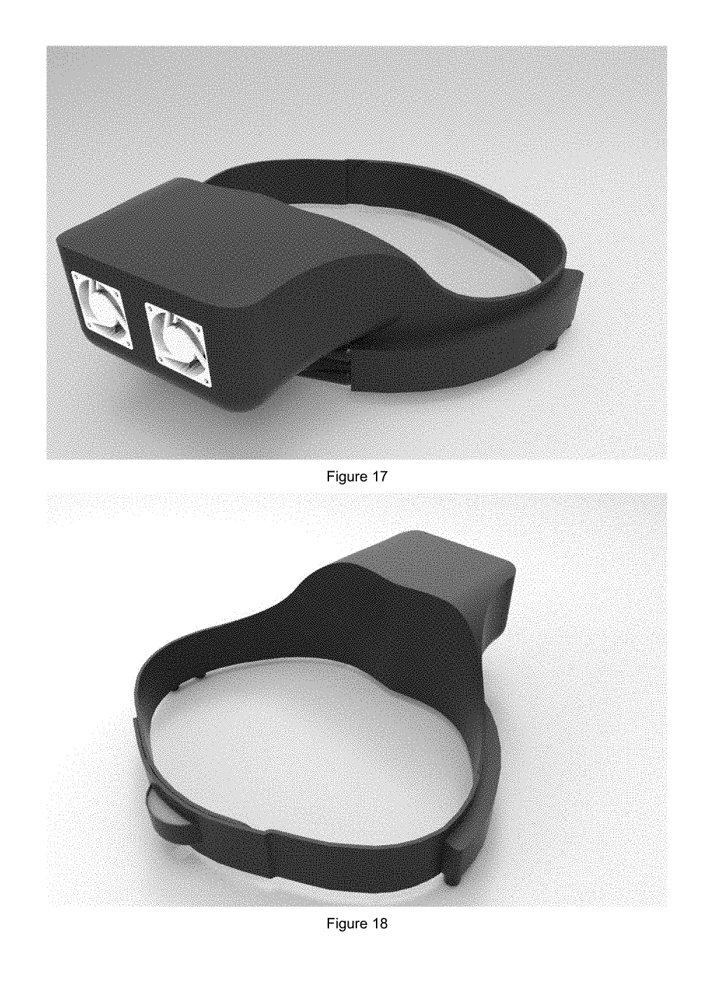

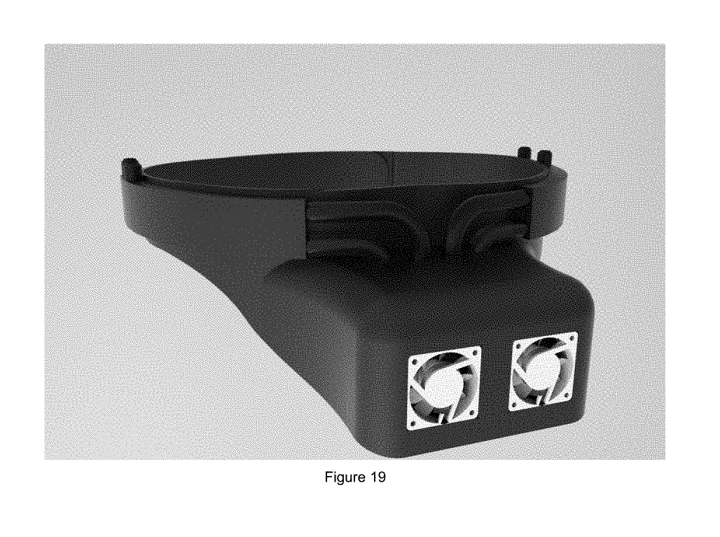

In a certain configuration the control unit, the thermoelectric module, the primary heat exchanger (6) and the pump are arranged in a portable module. Said portable module is exemplified by FIGS. 17 to 21.

The portable module may be arranged in the user's pelvic region such as a pouch, so that the subject can move freely during the temperature control. In addition, said module may be arranged as a backpack, wherein the subject carries it on the back or shoulder.

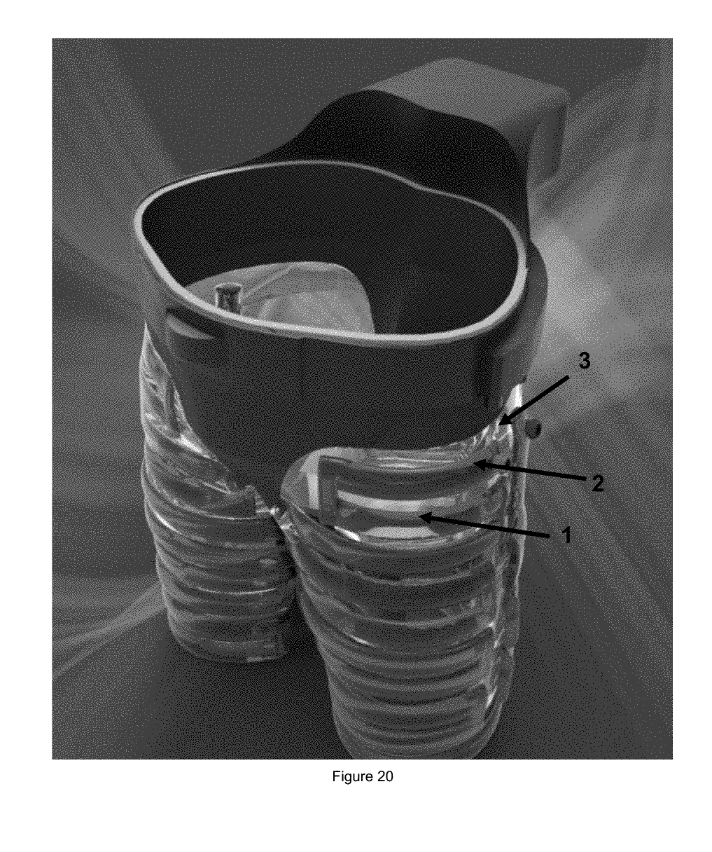

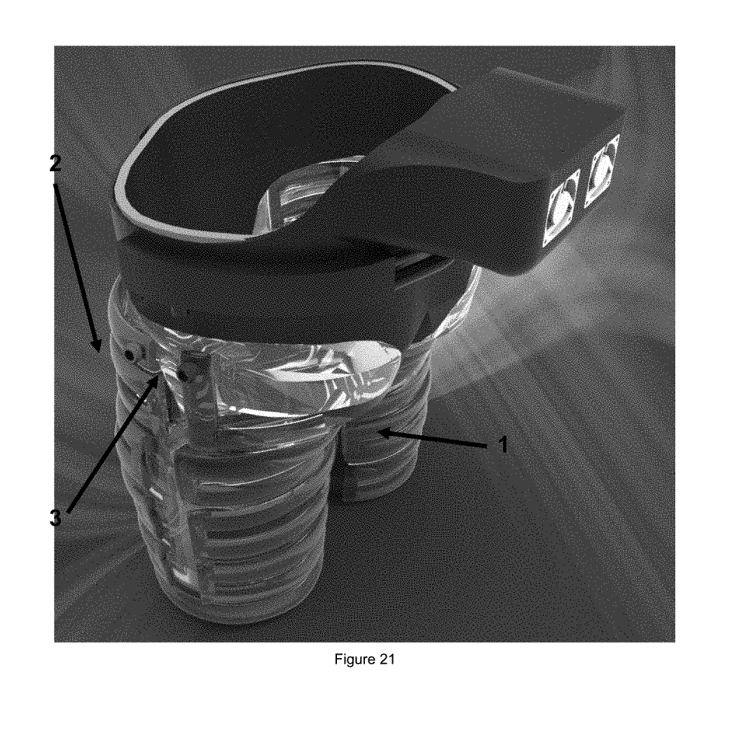

FIGS. 20 and 21 show one embodiment in which the primary heat exchanger (1) is used in conjunction with the portable module for temperature control of the user.

Thus, the user can control the temperature with autonomy during the practice of sports or daily activities and independently of third parties.

Therefore, the system can be used by patients, athletes, professionals and animals in a simple, autonomous, efficient and cost-effective way.

Those skilled in the art will appreciate the knowledge presented herein and may reproduce the invention in the embodiments presented and in other embodiments, falling within the scope of the appended claims.

1. A temperature control system for a cutaneous and/or muscular surface comprising at least:
a) a control unit;
b) a thermoelectric module;
c) a primary heat exchanger (6);
d) a pump; and
e) a secondary heat exchanger (1);
wherein,
the control unit is connected to the thermoelectric module;
thermoelectric module is associated to the primary heat exchanger (6); and
the pump is connected to the primary heat exchanger (6) and to the secondary heat exchanger (1).

2. The temperature control system for a cutaneous and/or muscular surface for a cutaneous and/or muscular surface according to claim 1, wherein the control unit comprises at least one electric current controller controlling thermoelectric module temperature.

3. The temperature control system for a cutaneous and/or muscular surface according to claim 2, wherein the thermoelectric module comprises a surface having heat absorption or heat dissipation, wherein absorption or dissipation is defined by the current controller of the control unit.

4. The temperature control system for a cutaneous and/or muscular surface according to claim 1, further comprising thermal association between the thermoelectric module and the primary heat exchanger (6).

5. The temperature control system for a cutaneous and/or muscular surface according to claim 1, wherein a surface of the thermoelectric module is connected to the primary heat exchanger (6).

6. The temperature control system for a cutaneous and/or muscular surface according to claim 1, further comprising an independent power supply associated to the control unit and the pump.

7. The temperature control system for a cutaneous and/or muscular surface according to claim 1, further comprising a temperature sensor.

8. The temperature control system for a cutaneous and/or muscular surface according to claim 1, further comprising fluid circulation in the primary heat exchanger (6) and secondary heat exchanger (1) through the pump.

9. The temperature control system for a cutaneous and/or muscular surface according to claim 1, wherein the system is a closed loop.

10. The temperature control system for a cutaneous and/or muscular surface according to claim 1, wherein the primary heat exchanger (6) and the secondary heat exchanger (1) are fluidly communicant.

11. The temperature control system for a cutaneous and/or muscular surface according to claim 1, further comprising fluid cooling and/or heating.

12. The temperature control system for a cutaneous and/or muscular surface according to claim 1, wherein the secondary heat exchanger (1) comprises an internal layer (1.1) and an external layer (1.2), wherein:
the internal layer (1.1) of the secondary heat exchanger (1) contacts the cutaneous and/or muscular surface.

13. The temperature control system for a cutaneous and/or muscular surface according to claim 12, wherein the internal layer (1.1) comprising comprises thermal conductor features.

14. The temperature control system for a cutaneous and/or muscular surface according to claim 12, wherein the external layer (1.2) is in contact with an external surface.

15. The temperature control system for a cutaneous and/or muscular surface according to claim 14, wherein the external layer (1.2) comprises thermal insulator features.

16. The temperature control system for a cutaneous and/or muscular surface according to the claim 14, wherein the external surface is defined by at least one of:
a) an external environment;
b) a thermal insulation layer (4); and
c) a heat sink.

17. The temperature control system for a cutaneous and/or muscular surface according to claim 12, wherein the internal layer (1.1) comprises a wide contact area regarding to the cutaneous and/or muscular surface.

18. The temperature control system for a cutaneous and/or muscular surface according to claim 1, wherein the secondary heat exchanger (1) comprises at least one of:
a) a serpentine;
b) a hive; and
c) any other tubular element for fluid circulation.

19. The temperature control system for a cutaneous and/or muscular surface according to claim 1, wherein the secondary heat exchanger (1) is associated with an insulator (4).

20. The temperature control system for a cutaneous and/or muscular surface according to claim 1, wherein the control unit, the thermoelectric module, the primary heat exchanger (6), and the pump are arranged in a portable module.

21. The temperature control system for a cutaneous and/or muscular surface according to claim 1, wherein the secondary heat exchanger (1) is associated with the lower and/or upper muscle group of a user.

22. The temperature control system for a cutaneous and/or muscular surface according to claim 21, further comprising a fabric layer between the secondary heat exchanger (1) and the cutaneous and/or muscular surface.

23. The temperature control system for a cutaneous and/or muscular surface according to claim 21, wherein the secondary heat exchanger (1) comprises a geometrical structure suitable with the muscular group.

24. The temperature control system for a cutaneous and/or muscular surface according to claim 12, wherein the secondary heat exchanger (1) is associated with a scalp of a user.

25. The temperature control system for a cutaneous and/or muscular surface according to claim 24, wherein the secondary heat exchanger (1) comprises a geometrical structure suitable with the scalp.

26. The temperature control system for a cutaneous and/or muscular surface according to claim 24, further comprising at least:
a) an adaption structure (4.2) associated with the secondary heat exchanger (1); and
b) an insulation layer (4.1) associated with the adaptation structure (4.2);
wherein,
the adaptation structure (4.2) and the insulation layer (4.1) each comprise geometrical structures suitable with the scalp.

27. The temperature control system for a cutaneous and/or muscular surface according to claim 26, further comprising a fluid inlet tube (2.1) associated with the secondary heat exchanger (1) and arranged in the adaptation structure (4.2).

28. The temperature control system for a cutaneous and/or muscular surface according to claim 27, wherein the fluid inlet tube (2.1) is thermally insulated.

29. The temperature control system for a cutaneous and/or muscular surface according to claim 12, wherein the secondary heat exchanger (1) is associated with a facial region of a user.

30. The temperature control system for a cutaneous and/or muscular surface according to claim 29, wherein the secondary heat exchanger (1) comprises a geometrical structure suitable with the user facial region.

31. The temperature control system for a cutaneous and/or muscular surface according to claim 29, further comprising a fabric layer between the secondary heat exchanger (1) and a facial surface of the facial region.

32. A method of temperature control for a cutaneous and/or muscular surface that is implemented in a temperature control system for a cutaneous and/or muscular surface, comprising at least the steps of:
a) adapting a contact area of a secondary heat exchanger (1) with the cutaneous and/or muscular surface;
b) providing a definition of fluid temperature;
c) providing current control in a thermoelectric module by a control unit;
d) pumping circulating fluid in a primary heat exchanger (6) and the secondary heat exchanger (1); and
e) recirculating the fluid of the secondary heat exchanger (1) to the primary exchanger (6);
wherein, said method is implemented in closed loop.

33. The method of temperature control for a cutaneous and/or muscular surface according to the claim 32, further comprising a step of providing a definition of exposure time of the cutaneous and/or muscular surface to the secondary heat exchanger (1).

34. The method of temperature control for a cutaneous and/or muscular surface according to claim 32, further comprising a step of providing temperature measurement for controlling user temperature.

35. The method of temperature control for a cutaneous and/or muscular surface according to claim 32, further comprising a step of providing a definition of the power supply, being at least one of:
a) supply medium independent from power grid; and
b) supply medium dependent from power grid;
wherein,
a switching is made between the supply medium.

36. The method of temperature control for a cutaneous and/or muscular surface according to claim 32, wherein a thermoelectric module heats/cools the primary heat exchanger (6).
